(19) United States
(12) Patent Application Publication
(10) Pub. No.: US 2009/0304802 A1
(43) Pub. Date: Dec. 10, 2009

(54) NASAL DELIVERY

(75) Inventors: Per Gisle Djupesland, Oslo (NO); Roderick Peter Hafner, Wiltshire (GB)

Correspondence Address: PROSKAUER ROSE LLP;PATENT DEPARTMENT, 1585 BROADWAY, NEW YORK, NY, 10036-8299, US

(73) Assignee: OptiNose AS, Oslo (NO)

(21) Appl. No.: 12/281,547

(22) PCT Filed: Mar. 5, 2007
(86) PCT No.: PCT/GB2007/000765

§ 371 Date: Jul. 17, 2009

(30) Foreign Application Priority Data
Mar. 3, 2006 (GB) 0604319.4

(51) Int. Cl. A61K 9/50; A61K 9/14; A61K 9/16; A61K 33/00; A61K 33/26; A61P 11/02; A61M 31/00

(52) U.S. Cl. 424/495; 424/489; 424/497; 424/490; 424/718; 424/646; 604/514; 604/187; 604/58

(57) ABSTRACT

A sustained release nasal formulation for delivery to a nasal cavity of a subject, wherein the formulation provides for sustained release of a substance, in particular nitric oxide (NO), to nasal mucosa within the nasal cavity so as to provide one or both of a therapeutic effect and promote normal nasal function, and a nasal delivery device and method relating thereto.

The present invention relates to the sustained release of substances, in particular nitric oxide (NO), in the nasal cavity of a subject, and more particularly the sustained release of nitric oxide to the nasal mucosa for providing one or both of a therapeutic effect and promoting normal nasal function.

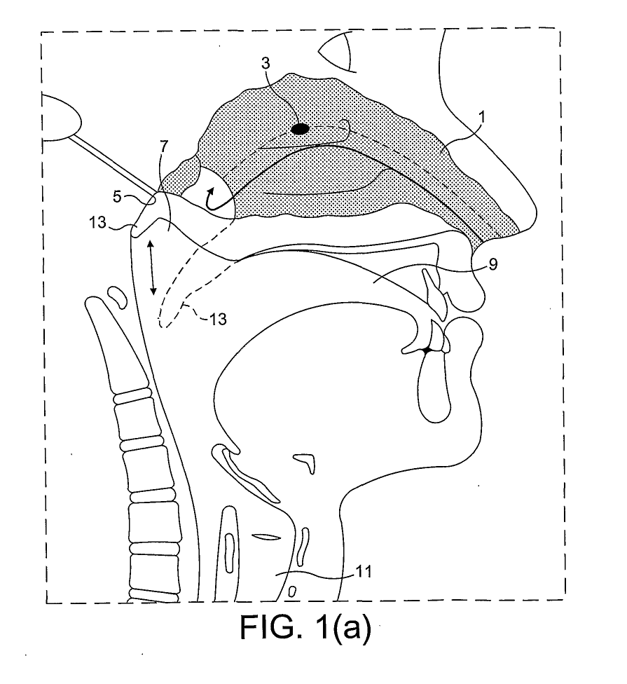

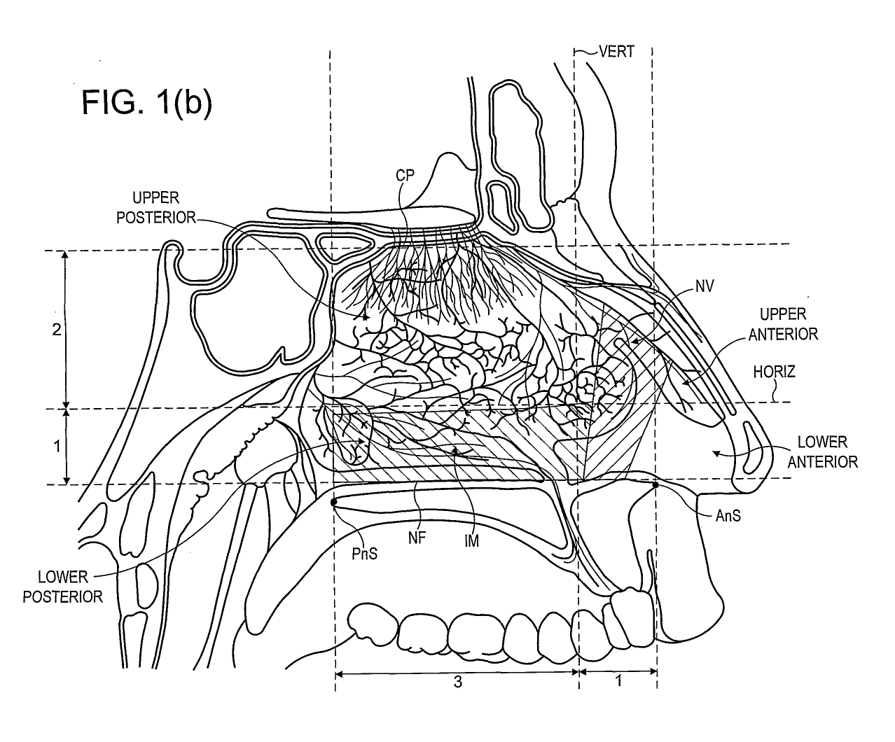

Referring to FIG. 1(a), the nasal airway 1 comprises the two nasal cavities separated by the nasal septum, which airway 1 includes numerous ostia, such as the paranasal sinus ostia 3 and the tubal ostia 5, and olfactory cells, and is lined by the nasal mucosa. The nasal airway 1 can communicate with the nasopharynx 7, the oral cavity 9 and the lower airway 11, with the nasal airway 1 being in selective communication with the anterior region of the nasopharynx 7 and the oral cavity 9 by opening and closing of the oropharyngeal velum 13. The velum 13, which is often referred to as the soft palate, is illustrated in solid line in the closed position, as achieved by providing a certain positive pressure in the oral cavity 9, such as achieved on exhalation through the oral cavity 9, and in dashed line in the open position.

As will be described in more detail hereinbelow, the present inventors have recognized that the sustained release of substances, in particular nitric oxide to the nasal mucosa in the posterior region of the nasal airway, and in particular the upper posterior region of the nasal airway, as illustrated in FIG. 1(b), relative to the anterior region of the nasal airway, is particularly advantageous.

The posterior region of the nasal airway is that region which is posterior of the nasal valve NV, as illustrated in FIG. 1(b). The nasal valve NV comprises the anterior bony cavum which contains inferior turbinate erectile tissue and septal erectile tissue, which are supported respectively by compliant ala tissue and the rigid cartilaginous septum (Cole). These elements combine to form a dynamic valve, which extends over several millimetres, that adjusts nasal airflow, and is stabilized by cartilage and bone, modulated by voluntary muscle and regulated by erectile tissue. The lumen of the nasal valve is the section of narrowest cross-sectional area between the posterior and anterior regions of the nasal airway, and is much longer and narrower dorsally than ventrally, and this lumen defines a triangular entrance which extends to the piriform region of the bony cavum. The nasal valve NV is lined in its anterior part with transitional epithelium, with a gradual transition posterior to respiratory epithelium. The nasal valve NV and anterior vestibule define roughly the anterior one-third of the nose.

The posterior region of the nasal airway is that region which is lined with respiratory epithelium, which is ciliated, and olfactory epithelium, which comprises nerves which extend downwards through the cribiform plate CP from the olfactory bulb, whereas the anterior region of the nasal airway is that region which is lined with squamous epithelium, which is not ciliated, and transitional epithelium. The olfactory epithelium extends on both the lateral and medial sides of the nasal airway, and typically extends downwards about 1.5 to 2.5 cm.

The upper posterior region is the region above the inferior meatus IM, as illustrated in FIG. 1(b), and encompasses the middle turbinate, the sinus ostia in infundibulum (ostia to maxillary, frontal and ethmoidal sinuses), the olfactory region, and the upper branches of the trigeminal nerve, and is that region which includes veins which drain to the venous sinuses that surround the brain.

As illustrated in FIG. 1(b), the posterior region of the nasal airway is the nasal region posterior of an imaginary vertical plane VERT which is located at a position corresponding to the lower angle of the anterior nasal aperture (aperture piriformis), which corresponds substantially to one-quarter of the distance between the anterior nasal spine AnS, which is a pointed projection at the anterior extremity of the intermaxillary suture, and the posterior nasal spine PnS, which is the sharp posterior extremity of the nasal crest of the hard palate and represents the transition between the nose and the nasopharynx, which corresponds to a distance posterior of the anterior nasal spine AnS of between about 13 mm and about 14 mm (Rosenberger defines the distance between the anterior nasal spine AnS and the posterior nasal spine PnS as being 56 mm in eighteen year old boys and 53.3 mm in eighteen year old girls).

As further illustrated in FIG. 1(b), the upper region of the nasal airway is an upper segment of the nasal airway which is bounded by the cribiform plate CP and a horizontal plane HORIZ which is located at a position corresponding to one-third of the distance between the nasal floor NF of the nasal airway and the cribiform plate CP, which corresponds to a height of typically between about 13 and about 19 mm above the nasal floor NF (Zacharek et al define the distance from the nasal floor NF to the cribiform plate CP as 46+/−4 mm).

The upper posterior region is thus that upper posterior region which is bounded by the above-defined vertical and horizontal planes VERT, HORIZ.

There has in recent years been considerable interest in the pharmaceutical application of nitric oxide.

Nitric oxide is a potent vasodilator, and has principally found application as a vasodilator in regulating local vascular resistance and blood flow.

Nitric oxide has also found application in the treatment of bacterial, viral or fungal conditions, as disclosed, for example, in WO-A-1995/022335 and WO-A-2001/053193.

In one aspect, it is an aim of the present invention to use a sustained release nitric oxide generator to provide for the sustained release of nitric oxide to the ciliated nasal mucosa to promote, and preferably restore, mucociliary function, such as in subjects suffering from rhinosinusitis and other infectious and inflammatory diseases, in particular of the sinuses, middle ears and adjacent structures.

In another aspect, it is an aim of the present invention to use a sustained release nitric oxide generator to provide for the sustained release of nitric oxide to the nasal mucosa to inhibit expression and liberation of pro-inflammatory cyokines and mediators.

In a further aspect, it is an aim of the present invention to use a sustained release nitric oxide generator to provide for the sustained release of nitric oxide to the nasal mucosa to at least reduce the replication of viruses, bacteria and fungi.

In a still further aspect, it is an aim of the present invention to use a sustained release nitric oxide generator to provide for the sustained release of nitric oxide to the nasal mucosa to prevent the progression of a simple, localized disease to otitis media, acute sinusitis, recurrent sinusitis or chronic rhinosinusitis, and also prevent the development or the severity of secondary complications as often seen in patients with asthma, cystic fibrosis, COPD, and a variety of hereditary and acquired immune deficiencies.

In a yet still further aspect, it is an aim of the present invention to use a sustained release nitric oxide generator to provide for the sustained release of nitric oxide to the nasal mucosa to provide therapeutic benefits in subjects with rhinosinusitis, polyposis, acute and recurrent sinusitis, common cold, cystic fibrosis and other infectious or inflammatory diseases, in particular of the sinuses, middle ears and adjacent organs and structures, such as through promoting mucociliary clearance in the ciliated nasal mucosa, inhibiting the expression and liberation of pro-inflammatory cyokines and mediators and reducing the replication of viruses, bacteria and fungi.

Mucociliary clearance is known to be depressed in subjects with nasal conditions, in particular rhinosinusitis, polyposis, acute sinusitis and common cold, and no therapeutic products exist to promote mucociliary activity.

Nitric oxide is a known up-regulator of ciliary activity, but, as mentioned hereinabove, is a potent vasodilator with systemic effects on the lungs and the heart. The administration of nitric oxide thus requires that the intake of pharmacological concentrations into the lungs be avoided, and, as such, the breathing of nitric oxide through the nose is not a viable therapeutic route.

The present inventors have recognized that, by formulating nitric oxide generators in a sustained release formulation, it is possible to avoid or at least reduce the likelihood of systemic side effects and also achieve a prolonged, sustained delivery of an effective concentration of nitric oxide in the nasal mucosa.

Nitric oxide generators have to date principally found application in achieving rapid vasodilation, and, as will be appreciated, this requirement is entirely contrary to that of the present invention, which requires the sustained release of nitric oxide at low concentrations.

In addition, because mucociliary clearance is rapid in healthy subjects, there are no intranasal sustained-release formulations, as these formulations would be rapidly cleared from the nose, typically in 10 to 15 minutes, and before the contained substance is released.

However, the present inventors have recognized that sustained release formulations can be delivered to subjects who have reduced mucociliary function, in particular patient groups who suffer from ciliostasis, and also that sustained release formulations can be delivered to the non-ciliated mucosa of the nasal cavity for therapeutic application.

In one aspect the present invention provides a sustained release nasal formulation for delivery to a nasal cavity of a subject, wherein the formulation provides for sustained release of a substance, in particular nitric oxide, to nasal mucosa within the nasal cavity.

In another aspect the present invention provides a method of providing for sustained release of a substance, in particular nitric oxide, to nasal mucosa within a nasal cavity of a subject, the method comprising the steps of: fitting a nosepiece unit to one nostril of a subject, the nosepiece unit including a nosepiece which is inserted into the one nostril of a subject and a nozzle through which a sustained release formulation is delivered to the respective nasal cavity; and delivering a sustained release formulation from the nozzle to the nasal cavity of the subject, wherein the formulation provides for sustained release of a substance to nasal mucosa within the nasal cavity.

In a further aspect the present invention provides a nasal delivery device for delivering a sustained release formulation to a nasal cavity of a subject, which provides for sustained release of a substance, in particular nitric oxide, to nasal mucosa within the nasal cavity, the delivery device comprising: a nosepiece unit including a nosepiece for fitting to a nostril of a subject and a nozzle through which the formulation is in use delivered to the respective nasal cavity; and a delivery unit for delivering the formulation through the nozzle of the nosepiece.

Nitric oxide generators include sodium nitroprusside, isosorbide dinitrate and glyceryl trinitrate, which release nitric oxide rapidly following application to biological surfaces.

Where delivered at high concentrations, subjects could suffer from transient, potentially fatal, systemic side effects. By formulating nitric oxide generators into sustained release formulations, it is possible both to reduce the likelihood of systemic side effects and prolong an effective concentration of nitric oxide in the nasal mucosa, and in one embodiment maintain a therapeutically-effective concentration of nitric oxide in the nasal mucosa.

In one embodiment the sustained release formulation is formulated as a liquid, typically a viscous liquid, such as a gel, and in particular a hydrogel.

In another embodiment the sustained release formulation is formulated as a powder, in particular a micropowder, such as loaded microspheres or coated microparticles.

In one embodiment the powders are fabricated by spray drying or freeze drying.

In one embodiment the sustained release formulation comprises polymer-coated microparticles, where the polymer is selected from one or more of ethylcellulose, methacrylic acid-methyl methacrylate copolymer, ethyl acrylate-methyl methacrylate-trimethylammonioethyl methacrylate chloride copolymer, hydroxypropyl methylcellulose phthalate, hydroxypropyl methylcellulose acetate succinate, carboxymethyl ethylcellulose and cellulose acetate phthalate.

In another embodiment the sustained release formulation comprises microparticles of a lipid structural matrix which encapsulates the substance or a substance-generating agent. In one embodiment the lipid matrix comprises a multilamellar structure of lipid bilayers having lipid chains ordered in an LβL phase, and the lipid matrix at least partially encapsulates the substance or substance-generating agent at a bilayer interface formed between head groups of adjacent lipid layers. In this embodiment the microparticles are prepared by heating a precursor formulation comprising a solvent, matrix-forming excipient and a substance or substance-generating agent to a temperature above the liquid-crystalline transition temperature Tc of the matrix-forming excipient and below the melting or denaturation point of the substance or substance-generating agent, and the solvent is then removed to form microparticles with partially encapsulated substance or substance-generating agent.

In one embodiment the thickness of the coating at least in part determines the time of release following administration, and a desired release profile is achieved by control of the particle size distribution and the thickness of the coated particles.

In one embodiment the powder comprises a substantial fraction of particles having sizes over the entire range of from about 1 μm to about 100 μm, preferably a major fraction of particles having a size greater than about 20 μm, and more preferably a major fraction of particles having a size greater than about 50 μm.

In one embodiment the sustained release formulation provides for the release of the nitric oxide generator at such a rate that the local concentration of nitric oxide does not cause a significant change in the diastolic blood pressure, and preferably a change in the diastolic blood pressure of not more than about 20 mm Hg.

In one embodiment the nosepiece is configured to extend into the nasal valve.

In one embodiment the subject exhales, preferably through a mouthpiece unit such as to cause closure of the oropharyngeal velum of the subject.

In one embodiment the nosepiece is fluidly connected to the mouthpiece unit, such that exhaled air from an exhalation breath is delivered through the nosepiece.

In one embodiment a gas flow, separate to an exhaled air flow from an exhalation breath of the subject, is delivered through the nosepiece.

In one embodiment at least 50%, preferably at least 55%, more preferably at least 60%, still more preferably at least 65% and yet more preferably at least 70% of the formulation as initially deposited in the nasal airway is deposited in a region of the nasal cavity which is posterior of the nasal valve.

In one embodiment the nosepiece is configured such as to obstruct the nasal valve.

In one embodiment the nosepiece is configured such as to close the nasal valve, and thereby prevent deposition of the formulation anteriorly of the same.

In one embodiment at least 30%, preferably at least 35%, more preferably at least 40%, still more preferably at least 45% and yet more preferably at least 50% of the formulation as initially deposited in the nasal cavity is deposited in an upper posterior region of the nasal cavity which is posterior of the nasal valve and above the inferior meatus.

In one embodiment the nosepiece unit includes a further nosepiece, and the further nosepiece is fitted to the other nostril of the subject, such as to at least partially obstruct the same.

In one embodiment the further nosepiece closes the other nostril.

In another embodiment the delivery device further comprises: a further nosepiece unit for fitting to the other nostril of the subject, the further nosepiece unit including a nosepiece for insertion into the other nostril of a subject and a nozzle through which the formulation is delivered to the respective nasal cavity.

In one embodiment the formulation provides for an effective concentration in the nasal mucosa for a period greater than 30 minutes, preferably for a period greater than 1 hour, more preferably for a period greater than 2 hours, still more preferably for a period greater than 4 hours, yet more preferably for a period greater than 6 hours, yet still more preferably for a period greater than 12 hours, and yet further more preferably for a period greater than 24 hours.

Preferred embodiments of the present invention will now be described hereinbelow by way of example only with reference to the accompanying drawings, in which:

FIG. 1(a) schematically illustrates the anatomy of the upper respiratory tract of a human subject;

FIG. 1(b) illustrates the segmentation of a nasal cavity in accordance with a preferred embodiment of the present invention;

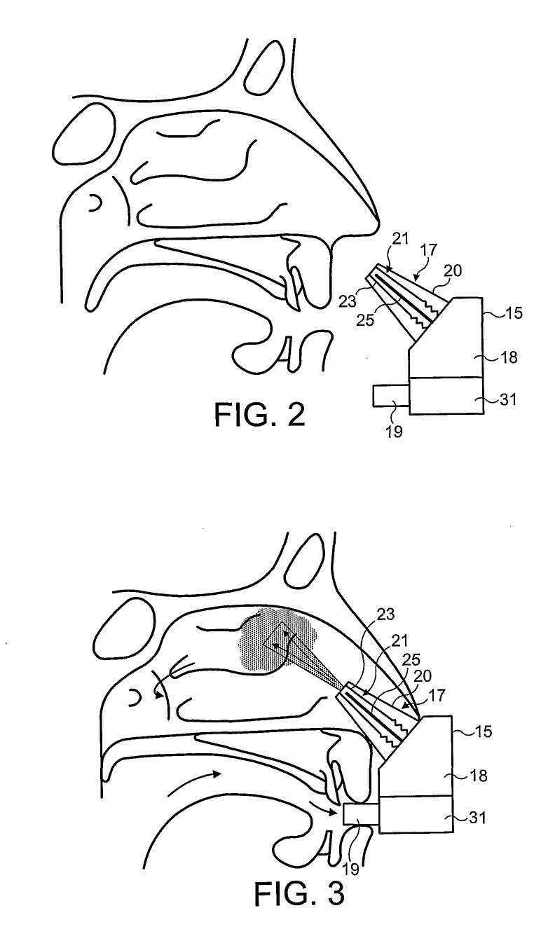

FIG. 2 illustrates a nasal delivery device in accordance with a first embodiment of the present invention;

FIG. 3 illustrates the nasal delivery device of FIG. 2, where operative in delivering formulation to the nasal cavity of the subject;

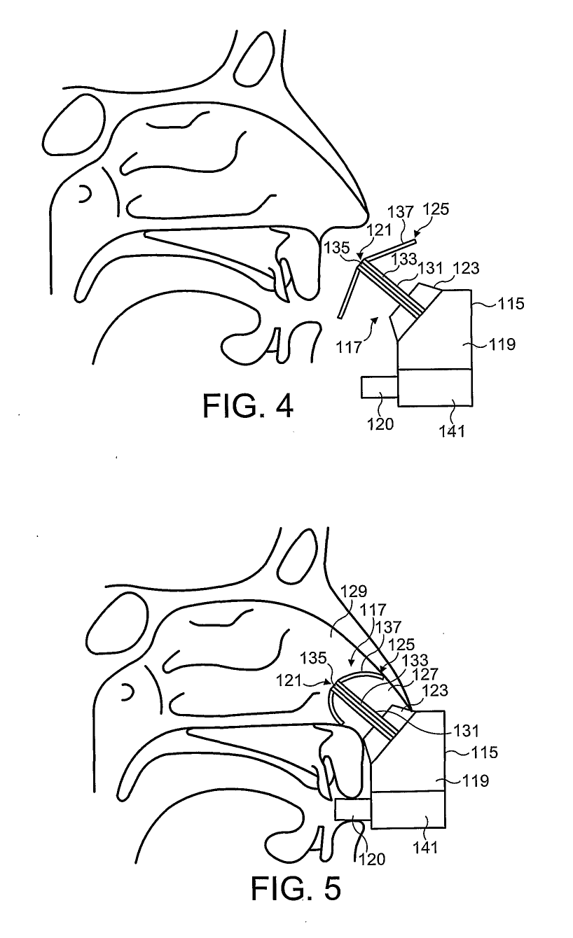

FIG. 4 illustrates a nasal delivery device in accordance with a second embodiment of the present invention, prior to insertion into the nasal cavity of the subject;

FIG. 5 illustrates the nasal delivery device of FIG. 4, following insertion into the nasal cavity of the subject; and

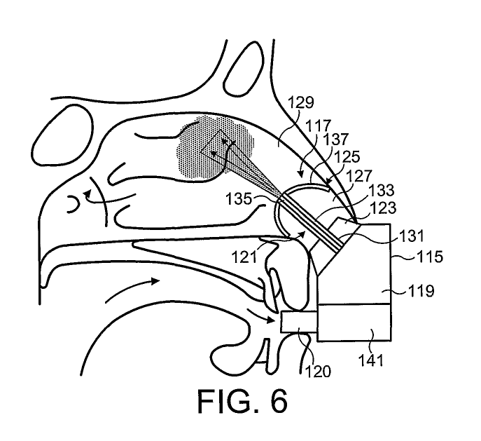

FIG. 6 illustrates the nasal delivery device of FIG. 4, where operative in delivering formulation to the nasal cavity of the subject.

FIGS. 2 and 3 illustrate a nasal delivery device in accordance with a first embodiment of the present invention.

The delivery device comprises a housing 15, a nosepiece unit 17 for fitting in a nasal passage of a subject, a supply unit 18 for delivering formulation, in this embodiment a sustained release formulation of a nitric oxide generator, to the nosepiece unit 17, and a mouthpiece 19 through which the subject exhales to actuate the delivery device.

The nosepiece unit 17 comprises a nosepiece 20, in this embodiment a frusto-conical element, for guiding the nosepiece unit 17 into a nasal passage of the subject and providing a fluid-tight seal With the nares of the nostril, and an outlet unit 21 for delivering formulation to a posterior region of the nasal passage of the subject.

In this embodiment the outlet unit 21 comprises a delivery channel 23 which is in fluid communication with the mouthpiece 19 such that an air flow is delivered into and through the nasal airway of the subject on exhalation by the subject through the mouthpiece 19, and a nozzle 25 which is in fluid communication with the supply unit 18 and provides for delivery of the formulation into the nasal passage of the subject.

In this embodiment the supply unit 18 comprises a mechanical delivery pump, in particular a liquid delivery pump or a powder delivery pump, which delivers metered doses of the formulation, on actuation thereof.

In an alternative embodiment the supply unit 18 could comprise a dry powder delivery unit which delivers metered doses of the formulation, as a dry powder, on actuation thereof. In one embodiment the supply unit 18 could provide for delivery of the formulation from a capsule.

In another alternative embodiment the supply unit 18 could comprise an aerosol canister which delivers metered volumes of a propellant, preferably a hydrofluoroalkane (HFA) propellant or the like, containing the formulation, either as a suspension or solution.

In yet another alternative embodiment the supply unit 18 could comprise a nebulizer which delivers metered doses of the formulation, as an aerosol spray, on actuation thereof.

In this embodiment the nozzle 25 is configured to deliver a significant fraction of the formulation to the upper posterior region of the nasal passage, here an initial deposition of greater than 30% of the delivered dose, preferably at least 35%, more preferably at least 40%, still more preferably at least 45% and yet more preferably at least 50%.

In one embodiment the delivery device is configured to deliver at least 50%, preferably at least 55%, more preferably at least 60%, still more preferably at least 65% and yet more preferably at least 70% of the formulation as initially deposited in the nasal airway is deposited in a region of the nasal cavity which is posterior of the nasal valve.

In this embodiment the nozzle 25 is configured to deliver the formulation as an aerosol spray.

In an alternative embodiment the nozzle 25 could be configured to deliver the formulation as a jet, for example, as a column of liquid or powder. In delivering the formulation as a jet, the formulation can be more readily targeted at a specific region within the posterior region of the nasal passage.

In this embodiment the supply unit 18 is a multi-dose unit for delivering a plurality of metered doses of the formulation. In another embodiment the supply unit 18 could be a single-dose unit for delivering a single metered dose of the formulation.

The supply unit 18 is pre-primeable, in this embodiment by loading a resilient element, and includes a breath-actuated release mechanism 31 which, when triggered, releases the resilient element and actuates the supply unit 18 to deliver a metered dose of the formulation through the nozzle 25.

In this embodiment the release mechanism 31 is configured to cause actuation of the supply unit 18 on generation of a predetermined pressure at the delivery channel 23.

In an alternative embodiment the release mechanism 31 could be configured to cause actuation of the supply unit 18 on generation of a predetermined flow rate through the delivery channel 23.

In this embodiment the nitric oxide generator comprises at least one of sodium nitroprusside, isosorbide dinitrate and glyceryl trinitrate, which release nitric oxide rapidly following application to biological surfaces.

In this embodiment the sustained release formulation is formulated as a liquid, typically a viscous liquid, such as a gel, and in particular a hydrogel.

In another embodiment the sustained release formulation could be formulated as a powder, in particular a micropowder, such as loaded microspheres or coated microparticles.

In one embodiment the powders are fabricated by spray drying or freeze drying.

In one embodiment the sustained release formulation comprises polymer-coated microparticles, where the polymer is selected from one or more of ethylcellulose, methacrylic acid-methyl methacrylate copolymer, ethyl acrylate-methyl methacrylate-trimethylammonioethyl methacrylate chloride copolymer, hydroxypropyl methylcellulose phthalate, hydroxypropyl methylcellulose acetate succinate, carboxymethyl ethylcellulose and cellulose acetate phthalate.

In another embodiment the sustained release formulation comprises microparticles of a lipid structural matrix which encapsulates the substance or a substance-generating agent. In one embodiment the lipid matrix comprises a multilamellar structure of lipid bilayers having lipid chains ordered in an LβL phase, and the lipid matrix at least partially encapsulates the substance or substance-generating agent at a bilayer interface formed between head groups of adjacent lipid layers. In this embodiment the microparticles are prepared by heating a precursor formulation comprising a solvent, matrix-forming excipient and a substance or substance-generating agent to a temperature above the liquid-crystalline transition temperature Tc of the matrix-forming excipient and below the melting or denaturation point of the substance or substance-generating agent, and the solvent is then removed to form microparticles with partially encapsulated substance or substance-generating agent.

In one embodiment the powder comprises a substantial fraction of particles having sizes over the entire range of from about 1 μm to about 100 μm, preferably a major fraction of particles having a size greater than about 20 μm, and more preferably a major fraction of particles having a size greater than about 50 μm.

Operation of the delivery device will now be described hereinbelow with reference to FIG. 3 of the accompanying drawings.

The nosepiece unit 17 is first inserted into one of the nasal passages of a subject until the nosepiece 20 abuts the nares of the nostril such as to establish a fluid-tight seal therewith, at which point the distal end of the outlet unit 21 extends about 2 cm into the nasal passage of the subject, and the mouthpiece 19 is gripped in the lips of the subject.

The subject then begins to exhale through the mouthpiece 19, which exhalation acts to close the oropharyngeal velum of the subject and drive an air flow through the delivery channel 23 of the outlet unit 21, with the air flow passing into the one nasal passage, around the posterior margin of the nasal septum and out of the other nasal passage, thereby achieving a bi-directional air flow through the nasal airway of the subject, as disclosed in the applicant's earlier WO-A-2000/051672, the content of which is incorporated herein by reference.

In this embodiment, when the pressure developed at the delivery channel 23 reaches a predetermined value, the release mechanism 31 is triggered to actuate the supply unit 18 to deliver a metered dose of the formulation to the nozzle 25 and into the nasal passage of the subject. The delivered formulation is deposited on the nasal mucosa.

As will be appreciated, this mode of delivery advantageously provides for the delivery of the formulation while the velum is closed, and thus prevents the inhalation of the formulation. As described hereinabove, nitric oxide has potentially dangerous systemic side effects which could arise as a result of inhalation of the delivered formulation.

In an alternative embodiment the release mechanism 31 could be triggered in response to the generation of a predetermined flow rate through the delivery channel 23.

In this embodiment, where the delivery device is a multi-dose device, the device is ready for further use following priming of the supply unit 18.

FIGS. 4 to 6 illustrate a nasal delivery device in accordance with a second embodiment of the present invention.

The delivery device comprises a housing 115, a nosepiece unit 117 for fitting in a nasal cavity of a subject through which the formulation is delivered to the nasal cavity, a supply unit 119 which is actuatable to deliver a metered dose of the formulation, in this embodiment a sustained release formulation of a nitric oxide generator, to the nosepiece unit 117, and a mouthpiece 120 through which the subject exhales to actuate the delivery device.

The nosepiece unit 117 comprises an outlet unit 121 which extends into the nasal cavity into which the nosepiece unit 117 is inserted, a first nosepiece member 123, in this embodiment a frusto-conical element, which is disposed to one, proximal end of the outlet unit 121 and is configured to obstruct, in this embodiment close, the nostril into which the nosepiece unit 117 is inserted, a second nosepiece member 125 which is disposed to the other, distal end of the outlet unit 121 and is configured to obstruct, in this embodiment close, the nasal cavity at a position therealong, in this embodiment at a position corresponding substantially to the nasal valve, such as to partition the nasal cavity into a first, anterior nasal section 127 between the first and second nosepiece members 123, 125, which corresponds in volume to about one-third of the nasal cavity, and a second, posterior nasal section 129, which corresponds in volume to about the remaining two-thirds of the nasal cavity, as illustrated in FIG. 5.

The outlet unit 121 comprises a support member 131, in this embodiment a narrow, elongate element, to which the first and second nosepiece members 123, 125 are supported, a delivery channel 133 which is in fluid communication with the mouthpiece 120 such that an air flow is delivered into and through the nasal airway of the subject on exhalation by the subject through the mouthpiece 120, and a nozzle 135 at the distal end thereof for delivering the formulation to the posterior nasal section 129 of the nasal cavity.

In this embodiment the nozzle 135 is configured to provide an aerosol spray. In an alternative embodiment, for the delivery of a liquid, the nozzle 135 could be configured to deliver a liquid jet as a column of liquid.

In this embodiment the nozzle 135 is configured to deliver a significant fraction of the formulation to the upper posterior region of the nasal passage, here an initial deposition of greater than 30% of the delivered dose, preferably at least 35%, more preferably at least 40%, still more preferably at least 45% and yet more preferably at least 50%.

In this embodiment the second nosepiece member 125 comprises a resilient element 137, here in the form of an annular skirt, which through its resilience acts to expand to obstruct the nasal cavity in partitioning the same. In an alternative embodiment the resilient element 137 could take the form of laterally-directed wings, which together act to obstruct the nasal cavity in partitioning the same.

In this embodiment the supply unit 119 comprises a mechanical delivery pump, in particular a liquid delivery pump or a powder delivery pump, which delivers metered doses of the formulation on actuation thereof.

In an alternative embodiment the supply unit 119 could comprise a dry powder delivery unit which delivers metered doses of the formulation, as a dry powder, on actuation thereof.

In another alternative embodiment the supply unit 119 could comprise an aerosol canister which delivers metered volumes of a propellant, preferably a hydrofluoroalkane (HFA) propellant or the like, containing the formulation, either as a suspension or solution.

In yet another alternative embodiment the supply unit 119 could comprise a nebulizer which delivers metered doses of the formulation, as an aerosol spray, on actuation thereof.

In this embodiment the supply unit 119 is a multi-dose unit for delivering a plurality of metered doses of the formulation. In another embodiment the supply unit 119 could be a single-dose unit for delivering a single metered dose of the formulation.

In this embodiment the supply unit 119 is pre-primeable, here by loading a resilient element, and includes a release mechanism 141 which, when triggered, releases the resilient element and actuates the supply unit 119 to deliver a metered dose of the formulation through the nozzle 135 of the outlet unit 121.

In this embodiment the nitric oxide generator comprises at least one of sodium nitroprusside, isosorbide dinitrate and glyceryl trinitrate, which release nitric oxide rapidly following application to biological surfaces.

In this embodiment the sustained release formulation is formulated as a liquid, typically a viscous liquid, such as a gel, and in particular a hydrogel.

In another embodiment the sustained release formulation could be formulated as a powder, in particular a micropowder, such as loaded microspheres or coated microparticles.

In one embodiment the powders are fabricated by spray drying or freeze drying.

In one embodiment the sustained release formulation comprises polymer-coated microparticles, where the polymer is selected from one or more of ethylcellulose, methacrylic acid-methyl methacrylate copolymer, ethyl acrylate-methyl methacrylate-trimethylammonioethyl methacrylate chloride copolymer, hydroxypropyl methylcellulose phthalate, hydroxypropyl methylcellulose acetate succinate, carboxymethyl ethylcellulose and cellulose acetate phthalate.

In another embodiment the sustained release formulation comprises microparticles of a lipid structural matrix which encapsulates the substance or a substance-generating agent. In one embodiment the lipid matrix comprises a multilamellar structure of lipid bilayers having lipid chains ordered in an LβL phase, and the lipid matrix at least partially encapsulates the substance or substance-generating agent at a bilayer interface formed between head groups of adjacent lipid layers. In this embodiment the microparticles are prepared by heating a precursor formulation comprising a solvent, matrix-forming excipient and a substance or substance-generating agent to a temperature above the liquid-crystalline transition temperature Tc of the matrix-forming excipient and below the melting or denaturation point of the substance or substance-generating agent, and the solvent is then removed to form microparticles with partially encapsulated substance or substance-generating agent.

In one embodiment the powder comprises a substantial fraction of particles having sizes over the entire range of from about 1 μm to about 100 μm, preferably a major fraction of particles having a size greater than about 20 μm, and more preferably a major fraction of particles having a size greater than about 50 μm.

Operation of the delivery device will now be described hereinbelow with reference to FIGS. 5 and 6 of the accompanying drawings.

Referring to FIG. 5, the nosepiece unit 117 is first inserted into one of the nasal cavities of a subject until the first nosepiece member 123 abuts the nares of the nostril. When inserted, the distal end of the outlet unit 121 extends about 2 cm into the nasal cavity of the subject, which corresponds in position to that of the nasal valve, and the second nosepiece member 125 acts to obstruct the nasal cavity at that point such as to partition the nasal cavity.

The subject then begins to exhale through the mouthpiece 120, which exhalation acts to close the oropharyngeal velum of the subject and drive an air flow through the delivery channel 133 of the outlet unit 121, with the air flow passing into the one nasal passage, around the posterior margin of the nasal septum and out of the other nasal passage, thereby achieving a bi-directional air flow through the nasal airway of the subject, as disclosed in the applicant's earlier WO-A-2000/051672, the content of which is incorporated herein by reference.

In this embodiment, when the pressure developed at the delivery channel 133 reaches a predetermined value, the release mechanism 141 is triggered to actuate the supply unit 119 to deliver a metered dose of the formulation to the nozzle 135 and into the nasal passage of the subject, as illustrated in FIG. 6. The delivered formulation is deposited on the nasal mucosa.

In an alternative embodiment the release mechanism 141 could be triggered in response to the generation of a predetermined flow rate through the delivery channel 133.

The metered dose of the formulation, in this embodiment in the form of an aerosol spray, is confined to the posterior region 129 of the nasal cavity as defined beyond the second nosepiece member 125. As described hereinabove, the present inventors have recognized that the systemic effect of the delivered substance can be substantially avoided by preventing delivery to the anterior region 127 of the nasal cavity, and confining the delivered dose of the formulation to the posterior region 129 of the nasal cavity. As described hereinabove, nitric oxide has potentially dangerous systemic side effects.

Similarly to the other-described embodiment, this mode of delivery also advantageously provides for the delivery of the formulation while the velum is closed, and thus prevents the inhalation of the substance, which could give rise to serious systemic side effects.

In one embodiment, where the delivery device is a single-dose device, the device can be discarded.

In another embodiment, where the delivery device is a multi-dose device, the device is ready for further use following priming of the supply unit 119. In a preferred embodiment, where the nosepiece unit 117 is replaceable, the nosepiece unit 117 can be replaced with a new nosepiece unit 117.

Finally, it will be understood that the present invention has been described in its preferred embodiments and can be modified in many different ways without departing from the scope of the invention as defined by the appended claims.

For example, in one embodiment, the present invention extends to the sustained release of substances other than nitric oxide.

In preferred embodiments the delivery devices are configured to deliver an air flow through one nostril of a subject at such a pressure as to flow around the posterior margin of the nasal septum and out of the other nostril of the subject, thereby achieving bi-directional delivery through the nasal cavities as disclosed in WO-A-00/51672, the content of which is herein incorporated by reference. In alternative embodiments the delivery device could be configured to deliver an air flow at a reduced pressure which is not sufficient to achieve bi-directional delivery through the nasal cavities. Such embodiments are still advantageous as compared to known delivery devices in providing for velum closure and being capable of achieving targeted delivery.

### REFERENCES

1. Cole, P, The Respiratory Role of the Upper Airways, a selective clinical and pathophysiological review, 1993, Mosby-Year Book Inc., ISBN 155664-390-X.

2. Rosenberger, H, Growth and Development of the Naso-Respiratory Area in Childhood, PhD Thesis, Laboratory of Anatomy, School of Medicine, Western Reserve University, Presented to the Annual Meeting of the American Laryngological, Rhinological and Otological Society, Charleston, S.C., USA, 1934.

3. Zacharek, M A et al, Sagittal and Coronal Dimensions of the Ethmoid Roof: A Radioanatomic Study, Am J Rhinol 2005, Vol 19, pages 348 to 352.

1. A sustained release nasal formulation for delivery to a nasal cavity of a subject, wherein the formulation provides for sustained release of a substance to nasal mucosa within the nasal cavity.

2. The formulation of claim 1, wherein the formulation contains a substance-generating agent which generates the substance on exposure to the nasal mucosa.

3. The formulation of claim 1, where formulated as a liquid, such as a viscous liquid, a gel, such as a hydrogel, including a chitosan hydrogel, or a powder, such as a micropowder.

4. (canceled)

5. (canceled)

6. The formulation of claim 3, where formulated as microspheres or microparticles, such as coated microparticles.

7. The formulation of claim 6, where formulated as polymer-coated microparticles, wherein the polymer coating preferably comprises one or more of ethylcellulose, methacrylic acid-methyl methacrylate copolymer, ethyl acrylate-methyl methacrylate-trimethylammonioethyl methacrylate chloride copolymer, hydroxypropyl methylcellulose phthalate, hydroxypropyl methylcellulose acetate succinate, carboxymethyl ethylcellulose and cellulose acetate phthalate.

8. (canceled)

9. The formulation of claim 6, where formulated as microparticles of a lipid structural matrix which encapsulates the substance or a substance-generating agent.

10. The formulation of claim 3, wherein the powder comprises a significant fraction of particles having sizes over the entire range of from about 1 μm to about 100 μm, preferably the powder has a major fraction of particles having a size greater than about 20 μm, and preferably a major fraction of particles having a size greater than about 50 μm.

11. (canceled)

12. The formulation of claim 1, wherein the formulation provides for release of the substance at such a rate that the local concentration of the substance does not cause a significant change in the diastolic blood pressure, and preferably a change in the diastolic blood pressure of not more than about 20 mm Hg.

13. The formulation of claim 1, wherein the formulation provides for an effective concentration in the nasal mucosa for a period greater than 30 minutes, preferably for a period greater than 1 hour, more preferably for a period greater than 2 hours, still more preferably for a period greater than 4 hours, yet more preferably for a period greater than 6 hours, yet still more preferably for a period greater than 12 hours, and yet further more preferably for a period greater than 24 hours.

14. The formulation of claim 1, wherein the substance comprises nitric oxide.

15. The formulation of claim 14, wherein the formulation comprises one or more of sodium nitroprusside, isosorbide dinitrate and glyceryl trinitrate as a nitric oxide generator which generates nitric oxide on exposure to the nasal mucosa.

16. The formulation of claim 14, wherein the formulation provides for (i) the sustained release of nitric oxide to the ciliated nasal mucosa to promote, and preferably restore, mucociliary function, such as in subjects suffering from rhinosinusitis and other infectious and inflammatory diseases, in particular of the sinuses, middle ears and adjacent structures, (ii) the sustained release of nitric oxide to the nasal mucosa to inhibit expression and liberation of pro-inflammatory cyokines and mediators, (iii) the sustained release of nitric oxide to the nasal mucosa to at least reduce replication of one or more of viruses, bacteria and fungi, (iv) the sustained release of nitric oxide to the nasal mucosa to prevent progression of a localized disease, in particular to otitis media, acute sinusitis, recurrent sinusitis or chronic rhinosinusitis, (v) the sustained release of nitric oxide to the nasal mucosa to prevent development or reduce a severity of secondary complications, such as observed in subjects with asthma, cystic fibrosis, COPD and a variety of hereditary and acquired immune deficiencies, or (vi) the sustained release of nitric oxide to the nasal mucosa to provide therapeutic benefits in subjects with rhinosinusitis, polyposis, acute and recurrent sinusitis, common cold, cystic fibrosis and other infectious or inflammatory diseases, in particular of the sinuses, middle ears and adjacent organs and structures, such as through promoting mucociliary clearance in the ciliated nasal mucosa, inhibiting expression and liberation of pro-inflammatory cyokines and mediators and reducing replication of one or more of viruses, bacteria and fungi.

17. (canceled)

18. (canceled)

19. (canceled)

20. (canceled)

21. (canceled)

22. A method of providing for sustained release of a substance to nasal mucosa within a nasal cavity of a subject, the method comprising the steps of:
fitting a nosepiece unit to one nostril of a subject, the nosepiece unit including a nosepiece which is inserted into the one nostril of a subject and a nozzle through which a sustained release formulation is delivered to the respective nasal cavity; and
delivering a sustained release formulation from the nozzle to the nasal cavity of the subject, wherein the formulation provides for sustained release of a substance to nasal mucosa within the nasal cavity.

23. The method of claim 22, wherein the nosepiece is configured to extend into the nasal valve.

24. The method of claim 22, further comprising the step of:
the subject exhaling through a mouthpiece unit such as to cause closure of the oropharyngeal velum of the subject.

25. The method of claim 24, wherein the nosepiece is fluidly connected to the mouthpiece unit, such that exhaled air from an exhalation breath is delivered through the nosepiece, or further comprising the step of:
delivering a gas flow, separate to an exhaled air flow from an exhalation breath of the subject, through the nosepiece.

26. (canceled)

27. The method of claim 22, wherein the nozzle provides for the delivery of a single jet or a plurality of jets, wherein the one or more jets comprise a liquid jet or a powder jet.

28. (canceled)

29. (canceled)

30. (canceled)

31. The method of claim 22, wherein the nozzle provides for the delivery of an aerosol spray, wherein the aerosol spray comprises a liquid spray or a powder spray.

32. (canceled)

33. (canceled)

34. The method of claim 22, further comprising the step of:
(i) manually actuating the delivery unit; or
(ii) actuating the delivery unit in response to oral exhalation by the subject.

35. (canceled)

36. The method of claim 22, wherein at least 50%, preferably at least 55%, more preferably at least 60%, still more preferably at least 65% and yet more preferably at least 70% of the formulation as initially deposited in the nasal airway is deposited in a region of the nasal cavity which is posterior of the nasal valve.

37. The method of claim 36, wherein the nosepiece is configured such as to obstruct the nasal valve, and preferably the nosepiece is configured such as to close the nasal valve, and thereby prevent deposition of the formulation anteriorly of the same.

38. (canceled)

39. The method of claim 22, wherein at least 30%, preferably at least 35%, more preferably at least 40%, still more preferably at least 45% and yet more preferably at least 50% of the formulation as initially deposited in the nasal cavity is deposited in an upper posterior region of the nasal cavity which is posterior of the nasal valve and above the inferior meatus.

40. The method of claim 22, wherein the nosepiece unit includes a further nosepiece, and the nosepiece unit fitting step further comprises the step of:
fitting the further nosepiece to the other nostril of the subject, such as to at least partially obstruct the same, and preferably close the same.

41. (canceled)

42. The method of claim 22, further comprising the step of:
fitting a further nosepiece unit to the other nostril of the subject, the nosepiece unit including a nosepiece for insertion into the other nostril of a subject and a nozzle through which the formulation is delivered to the respective nasal cavity.

43. The method of claim 22, wherein the formulation contains a substance-generating agent which generates the substance on exposure to the nasal mucosa.

44. The method of claim 22, where formulated as a liquid, such as a viscous liquid, a gel, such as a hydrogel, including a chitosan hydrogel, or a powder, such as a micropowder.

45. (canceled)

46. (canceled)

47. The method of claim 44, where formulated as microspheres or microparticles, such as coated microparticles.

48. The method of claim 47, where formulated as polymer-coated microparticles, wherein the polymer coating preferably comprises one or more of ethylcellulose, methacrylic acid-methyl methacrylate copolymer, ethyl acrylate-methyl methacrylate-trimethylammonioethyl methacrylate chloride copolymer, hydroxypropyl methylcellulose phthalate, hydroxypropyl methylcellulose acetate succinate, carboxymethyl ethylcellulose and cellulose acetate phthalate.

49. (canceled)

50. The method of claim 47, where formulated as microparticles of a lipid structural matrix which encapsulates the substance or a substance-generating agent.

51. The method of claim 47, wherein the powder comprises a significant fraction of particles having sizes over the entire range of from about 1 μm to about 100 μm, preferably the powder has a major fraction of particles having a size greater than about 20 μm, and preferably a major fraction of particles having a size greater than about 50 μm.

52. (canceled)

53. The method of claim 22, wherein the formulation provides for release of the substance at such a rate that the local concentration of the substance does not cause a significant change in the diastolic blood pressure, and preferably a change in the diastolic blood pressure of not more than about 20 mm Hg.

54. The method of claim 22, wherein the formulation provides for an effective concentration in the nasal mucosa for a period greater than 30 minutes, preferably for a period greater than 1 hour, more preferably for a period greater than 2 hours, still more preferably for a period greater than 4 hours, yet more preferably for a period greater than 6 hours, yet still more preferably for a period greater than 12 hours, and yet further more preferably for a period greater than 24 hours.

55. The method of claim 22, wherein the substance comprises nitric oxide.

56. The method of claim 55, wherein the formulation comprises one or more of sodium nitroprusside, isosorbide dinitrate and glyceryl trinitrate as a nitric oxide generator which generates nitric oxide on exposure to the nasal mucosa.

57. The method of claim 55, wherein the formulation provides for the sustained release of nitric oxide to the ciliated nasal mucosa to promote, and preferably restore, mucociliary function, such as in subjects suffering from rhinosinusitis and other infectious and inflammatory diseases, in particular of the sinuses, middle ears and adjacent structures.

58. The method of claim 55, wherein the formulation provides for the sustained release of nitric oxide to the nasal mucosa to inhibit expression and liberation of pro-inflammatory cyokines and mediators.

59. The method of claim 55, wherein the formulation provides for the sustained release of nitric oxide to the nasal mucosa to at least reduce replication of one or more of viruses, bacteria and fungi.

60. The method of claim 55, wherein the formulation provides for the sustained release of nitric oxide to the nasal mucosa to prevent progression of a localized disease, in particular to otitis media, acute sinusitis, recurrent sinusitis or chronic rhinosinusitis, and preferably the formulation provides for the sustained release of nitric oxide to the nasal mucosa to prevent development or reduce a severity of secondary complications, such as observed in subjects with asthma, cystic fibrosis, COPD and a variety of hereditary and acquired immune deficiencies.

61. (canceled)

62. The method of claim 55, wherein the formulation provides for the sustained release of nitric oxide to the nasal mucosa to provide therapeutic benefits in subjects with rhinosinusitis, polyposis, acute and recurrent sinusitis, common cold, cystic fibrosis and other infectious or inflammatory diseases, in particular of the sinuses, middle ears and adjacent organs and structures, such as through promoting mucociliary clearance in the ciliated nasal mucosa, inhibiting expression and liberation of pro-inflammatory cyokines and mediators and reducing replication of one or more of viruses, bacteria and fungi.

63. A nasal delivery device for delivering a sustained release formulation to a nasal cavity of a subject, which provides for sustained release of a substance to nasal mucosa within the nasal cavity, the delivery device comprising:
a nosepiece unit including a nosepiece for fitting to a nostril of a subject and a nozzle through which the formulation is in use delivered to the respective nasal cavity; and
a delivery unit for delivering the formulation through the nozzle of the nosepiece.

64. The delivery device of claim 63, wherein the nosepiece is configured, when inserted into the nasal cavity, to extend into the nasal valve.

65. The delivery device of claim 63, further comprising:
a mouthpiece unit through which the subject in use exhales to cause closure of the oropharyngeal velum of the subject.

66. The delivery device of claim 65, further comprising:
(i) a flow channel fluidly connecting the nosepiece and the mouthpiece unit, whereby exhaled air from an exhalation breath is delivered through the nosepiece; or
(ii) a flow channel fluidly connected to the nosepiece through which a gas flow, separate to an exhaled air flow from an exhalation breath of the subject, is in use delivered to the nosepiece; and
a gas supply unit for supplying a gas flow to the flow channel.

67. (canceled)

68. The delivery device of claim 63, wherein the nozzle provides for the delivery of a single jet or a plurality of jets, wherein the one or more jets comprise a liquid jet or a powder jet.

69. (canceled)

70. (canceled)

71. (canceled)

72. The delivery device of any of claim 63, wherein the nozzle provides for the delivery of an aerosol spray, wherein the aerosol spray comprises a liquid spray or a powder spray.

73. (canceled)

74. (canceled)

75. The delivery device of claim 63, wherein the delivery unit is manually actuatable, or further comprising:
an actuation mechanism for actuating the delivery unit in response to oral exhalation by the subject, wherein the actuation mechanism is preferably configured such as to be actuated in response to generation of a predeterminable pressure in the nasal cavity.

76. (canceled)

77. (canceled)

78. The delivery device of claim 63, wherein the delivery device is configured such that at least 50%, preferably at least 55%, more preferably at least 60%, still more preferably at least 65% and yet more preferably at least 70% of the formulation as initially deposited in the nasal cavity is deposited in the region posterior of the nasal valve.

79. The delivery device of claim 78, wherein the nosepiece is configured such as to obstruct the nasal valve, and preferably close the nasal valve, and thereby prevent deposition of the formulation anteriorly of the same.

80. (canceled)

81. The delivery device of claim 63, wherein the delivery device is configured such that at least 30%, preferably at least 35%, more preferably at least 40%, still more preferably at least 45% and yet more preferably at least 50% of the formulation as initially deposited in the nasal cavity is deposited in the upper posterior region thereof.

82. The delivery device of claim 63, wherein the nosepiece unit includes a further nosepiece for fitting to the other nostril of the subject and at least partially obstructing the same, and preferably close the same.

83. (canceled)

84. The delivery device of claim 63, further comprising:
a further nosepiece unit including a nosepiece for fitting to the other nostril of the subject and a nozzle through which the formulation is in use delivered to the respective nasal cavity.

85. The delivery device of claim 63, wherein the formulation contains a substance-generating agent which generates the substance on exposure to the nasal mucosa.

86. The delivery device of claim 63, where formulated as a liquid, such as a viscous liquid, a gel, such as a hydrogel, including a chitosan hydro gel, or a powder, such as a micropowder.

87. (canceled)

88. (canceled)

89. The delivery device of claim 86, where formulated as microspheres or microparticles, such as coated microparticles.

90. The delivery device of claim 89, where formulated as polymer-coated microparticles, wherein the polymer coating preferably comprises one or more of ethylcellulose, methacrylic acid-methyl methacrylate copolymer, ethyl acrylate-methyl methacrylate-trimethylammonioethyl methacrylate chloride copolymer, hydroxypropyl methylcellulose phthalate, hydroxypropyl methylcellulose acetate succinate, carboxymethyl ethylcellulose and cellulose acetate phthalate.

91. (canceled)

92. The delivery device of claim 89, where formulated as microparticles of a lipid structural matrix which encapsulates the substance or a substance-generating agent.

93. The delivery device of claim 86, wherein the powder comprises a significant fraction of particles having sizes over the entire range of from about 1 μm to about 100 μm, preferably the powder has a major fraction of particles having a size greater than about 20 μm, and preferably a major fraction of particles having a size greater than about 50 μm.

94. (canceled)

95. The delivery device of claim 63, wherein the formulation provides for release of the substance at such a rate that the local concentration of the substance does not cause a significant change in the diastolic blood pressure, and preferably a change in the diastolic blood pressure of not more than about 20 mm Hg.

96. The delivery device of claim 63, wherein the formulation provides for an effective concentration in the nasal mucosa for a period greater than 30 minutes, preferably for a period greater than 1 hour, more preferably for a period greater than 2 hours, still more preferably for a period greater than 4 hours, yet more preferably for a period greater than 6 hours, yet still more preferably for a period greater than 12 hours, and yet further more preferably for a period greater than 24 hours.

97. The delivery device of claim 63, wherein the substance comprises nitric oxide.

98. The delivery device of claim 97, wherein the formulation comprises one or more of sodium nitroprusside, isosorbide dinitrate and glyceryl trinitrate as a nitric oxide generator which generates nitric oxide on exposure to the nasal mucosa.

99. The delivery device of claim 97, wherein the formulation provides for (i) the sustained release of nitric oxide to the ciliated nasal mucosa to promote, and preferably restore, mucociliary function, such as in subjects suffering from rhinosinusitis and other infectious and inflammatory diseases, in particular of the sinuses, middle ears and adjacent structures, (ii) the sustained release of nitric oxide to the nasal mucosa to inhibit expression and liberation of pro-inflammatory cyokines and mediators, (iii) the sustained release of nitric oxide to the nasal mucosa to at least reduce replication of one or more of viruses, bacteria and fungi, (iv) the sustained release of nitric oxide to the nasal mucosa to prevent progression of a localized disease, in particular to otitis media, acute sinusitis, recurrent sinusitis or chronic rhinosinusitis, (v) the sustained release of nitric oxide to the nasal mucosa to prevent development or reduce a severity of secondary complications, such as observed in subjects with asthma, cystic fibrosis, COPD and a variety of hereditary and acquired immune deficiencies, or (vi) the sustained release of nitric oxide to the nasal mucosa to provide therapeutic benefits in subjects with rhinosinusitis, polyposis, acute and recurrent sinusitis, common cold, cystic fibrosis and other infectious or inflammatory diseases, in particular of the sinuses, middle ears and adjacent organs and structures, such as through promoting mucociliary clearance in the ciliated nasal mucosa, inhibiting expression and liberation of pro-inflammatory cyokines and mediators and reducing replication of viruses, bacteria and fungi.

100. (canceled)

101. (canceled)

102. (canceled)

103. (canceled)

104. (canceled)
